(19) United States
(12) Patent Application Publication
(10) Pub. No.: US 2017/0044478 A1
(43) Pub. Date: Feb. 16, 2017

(54) METHOD AND SYSTEM FOR GROWING MICROALGAE IN EXPANDING SLOPED PONDS

(71) Applicant: GENERAL ATOMICS, San Diego, CA (US)

(72) Inventors: Kevin W. Downey, San Diego, CA (US); Kenneth R. Liberty, Poway, CA (US); David A. Hazlebeck, El Cajon, CA (US); Xiaoxi Wu, Encinitas, CA (US)

(21) Appl. No.: 15/258,785

(22) Filed: Sep. 7, 2016

Related U.S. Application Data
(63) Continuation-in-part of application No. 14/256,803, filed on Apr. 18, 2014, now abandoned.
Application No. 14/256,803 is a continuation of application No. 12/821,943, filed on Jun. 23, 2010, now abandoned.

(51) Int. Cl. C12M 1/00; C12M 1/34

(52) U.S. Cl. CPC C12M 23/18; C12M 41/46; C12M 29/00

(57) ABSTRACT

A system for growing an algal culture to create a biomass includes a plurality of linearly interconnected, sloped-gradient, gravity-driven, raceway ponds. Surface areas of the ponds are sequentially increased in accordance with a multiplier, with the pond surface area of the last raceway pond in the sequence being as large as fifty acres. For the present invention, a fluid transfer system connects each raceway pond with every other raceway pond in the system. Control over each individual raceway pond is provided to monitor and evaluate algal culture in the pond. Based on this evaluation, the fluid transfer system is activated to provide water, nutrients and other additives to maintain predetermined growth parameters for algae in each of the raceway ponds.

This application is a continuation-in-part of application Ser. No. 14/256,803, filed Apr. 18, 2014, which is currently pending, and which is a continuation of application Ser. No. 12/821,943, filed Jun. 23, 2010, which is now abandoned. The contents of application Ser. Nos. 14/256,803 and 12/821,943 are incorporated herein by reference.

The U.S. Government has a paid-up license in this invention and the right in limited circumstances to require the patent owner to license others on reasonable terms as provided for by the terms of Contract No. HR0011-09-C-0034 awarded by DARPA.

### FIELD OF THE INVENTION

The present invention pertains generally to systems and methods for growing algae. More particularly, the present invention pertains to the use of an expanding plug flow reactor concept to reduce the requirement of using expensive closed system bioreactors for growing algae. The present invention is particularly, but not exclusively, useful as a method for growing algae in an open system comprising an expanding plug flow reactor including gravity-driven raceway ponds which are controlled to maintain a constant high concentration of algae cells in each pond.

### BACKGROUND OF THE INVENTION

As worldwide petroleum deposits decrease, there is rising concern over shortages and the costs that are associated with the production of hydrocarbon products. As a result, alternatives to products that are currently processed from petroleum are being investigated. In this effort, biofuel such as biodiesel has been identified as a possible alternative to petroleum-based transportation fuels. In general, a biodiesel is a fuel comprised of mono-alkyl esters of long chain fatty acids derived from plant oils or animal fats. In industrial practice, biodiesel is created when plant oils or animal fats are reacted with an alcohol, such as methanol.

For plant-derived biofuel, solar energy is first transformed into chemical energy through photosynthesis. The chemical energy is then refined into a usable fuel. Currently, the process involved in creating biofuel from plant oils is expensive relative to the process of extracting and refining petroleum. It is possible, however, that the cost of processing a plant-derived biofuel could be reduced by maximizing the rate of growth of the plant source. Because algae is known to be one of the most efficient plants for converting solar energy into cell growth, it is of particular interest as a biofuel source. Importantly, the use of algae as a biofuel source presents no exceptional problems, i.e., biofuel can be processed from oil in algae as easily as from oils in land-based plants. Further, an algal biomass which is grown in accordance with the present invention is also useful for products such as (1) high-protein feedstock for aquaculture and animal feeds, and (2) oils/pigments for cosmetics, dyes, and nutraceuticals.

While algae can efficiently transform solar energy into chemical energy via a high rate of cell growth, it has been difficult to create environments in which algae cell growth rates are optimized. Currently, the production of biofuel from algae is limited by a failure to maximize algae cell growth. Specifically, the conditions necessary to facilitate a fast growth rate for algae cells in large-scale operations have been found to be expensive to create. For instance, while providing high rates of algae cell growth, closed sterile environments such as inoculant tanks and controlled photobioreactors are expensive to maintain and are severely limited in scale. On the other hand, outdoor large-scale open systems, such as open raceways, are typically plagued by contaminant organisms which compete with the selected algae cells for nutrients and sunlight and reduce the rate of algae cell growth. Specifically, these contaminants include non-selected, i.e., “weed”, algae, viruses, bacteria, and grazers. Until now, it has been virtually impossible to prevent contaminant organisms from causing microbial instability and reducing selected algae cell growth rates in open systems. In fact, standard open systems typically provide only one to two days of microbial stability.

In light of the above, it is an object of the present invention to provide a method for minimizing the need for closed system inoculation of algae cells in a biofuel production system. Another object of the present invention is to maximize the cell growth rate of selected algae cells in an open system. Another object of the present invention is to provide an expanding plug flow reactor for supporting logarithmic growth of algae cells. Another object of the present invention is to selectively pump medium into the expanding plug flow reactor to maintain a constant, high concentration of algae and a selected shallow depth of medium. Still another object of the present invention is to provide a method and system for growing selected algae cells in an open system in which contaminants cannot compete with the selected algae cells. Yet another object of the present invention is to provide a system and method for growing selected algae cells that is simple to implement, easy to use, and comparatively cost effective. Finally, another object of the present invention is to provide a methodology for sloped open pond expansion that is capable of supporting individual sloped pond sizes of up to 50 acres in wetted area.

### SUMMARY OF THE INVENTION

In accordance with the present invention, a system is provided for growing selected algae cells in a medium and for preventing the growth of contaminants in the medium. In this endeavor, the system relies on the initial use of a closed reactor to grow an inoculum of microalgae. Importantly, the closed reactor is five times smaller than those used in known algae production systems. Specifically, the closed reactor comprises 0.4% of the present system while closed reactors typically comprise about 2% of known systems. For purposes of the present invention, the closed reactor is a continuous flow reactor such as a photobioreactor. Further, the closed reactor is designed to grow the inoculum of microalgae to a full targeted concentration.

After the closed reactor grows microalgae to full concentration, the inoculum of microalgae is passed in an effluence to an open system. Specifically, the open system comprises an expanding plug flow reactor and a standard plug flow reactor. For the present invention, the expanding plug flow reactor continuously receives the effluence containing the inoculum of algae cells from the closed reactor. Further, the expanding plug flow reactor includes a conduit for continuously moving the effluence downstream under the influence of gravity with little back mixing. Preferably, the expanding plug flow reactor is an open raceway.

Structurally, the expanding plug flow reactor increases in width from its first end to its second end. Also, the expanding plug flow reactor is provided with a plurality of pumps along its length for introducing a growth medium to the conduit. Initially, the pumps feed the growth medium at a rate consistent with maintaining a high algal concentration. For purposes of the present invention, “high concentration” is defined as a range between about 0.5 grams and 1.0 gram per liter of fluid. Thereafter, as fluid evaporates and the algae cells grow, the pumps add growth medium to maintain the high concentration of algae at a near-constant level. Further, the growth medium includes the nutrients necessary to support the desired growth of the algae cells.

Importantly, the pumps are controlled in response to the growth rate of the algae cells, For instance, the algae growth rate may decrease due to a reduction in the amount of sunlight received and lower air temperatures. As a result, in order to ensure a high concentration of algae as the expanding plug flow reactor widens, the pumps will provide less medium. Therefore, the depth of the medium will decrease slightly, and the flow rate of the algae cells will decrease due to the increased laminar flow effects. However, with sufficient nutrients and the maintenance of a proper depth, the algae cells are able to grow sufficiently to remain at a high concentration as the expanding plug flow reactor widens. Because the selected algae is maintained at a high concentration, the nutrients provided in the growth medium are rapidly consumed by the selected algae. As a result, the time available for growth of contaminants is limited.

When the selected algae cells reach the end of the expanding plug flow reactor, they have reached the desired level of growth. Thereafter, the algae cells are transferred to a standard plug flow reactor. Typically, the standard plug flow reactor will have sufficient volume to accommodate an appropriate amount of algae culture from the downstream end of the expanding plug flow reactor. Further, a trigger medium may be fed into the standard plug flow reactor to activate production of oil in the algae cells. Alternatively, no medium may be fed into the standard plug flow reactor. In either case, the effect is to trigger oil production because algae cells will convert stored energy to oil when being starved of certain, or all, nutrients.

For an alternate embodiment of the present invention, a system for growing algae cells includes a plurality of open ponds. In combination, open ponds in this plurality are connected for selective fluid communication with each other, and they are arranged in sequence from a first upstream pond to a last downstream pond. In a variation from the expanded plug flow reactor (EPFR) described above, this alternate embodiment of the invention establishes each downstream pond with an exponentially greater surface area relative to its adjacent upstream pond.

Structurally, the alternate embodiment of the present invention includes a first transfer conduit for transferring inoculum from an inoculum source into the first upstream pond. A culture is thereby created for algae growth in the first upstream pond. A subsequent transfer of the culture can then be made from the first upstream pond to successive downstream ponds for further algae growth. For the present invention, such transfers are periodically accomplished in a controlled manner, and algae is allowed to grow for a predetermined time in each of the successive ponds. Eventually, fully grown algae cells are transferred from the last downstream pond to either an oil formation pond via a last transfer conduit if additional oil formation is desired, or directly to a harvesting system, if, for example, algal biomass is the desired end product.

Each open pond in the system, regardless of its relative size, will preferably have a fluid circulating device, such as a paddle wheel or circulation pump, that can be used to establish liquid flow in the pond. Smaller ponds will typically circulate media via paddle wheels, while larger ponds in the present invention will be sloped to provide gravity-driven mixing. Preferably, each pond will also have a medium addition conduit for adding medium into the culture in the pond. Further, as envisioned for the present invention, the transfer of culture from an upstream pond to its adjacent downstream pond can be accomplished in either of two ways. For one, each pond may include a transfer pump for transferring the culture downstream from the pond to its adjacent downstream pond. For another, the ponds can be terraced so that a gravity flow can be established from an upstream pond to a downstream pond.

As implied above, a fixed multiplier is determined to establish a ratio of the surface areas for adjacent ponds. More specifically, the surface area of each pond relative to the surface area of an adjacent upstream or downstream pond will be established by this multiplier. In practice, the value of the multiplier may vary from system to system. Specifically, in each case the multiplier will be determined by the established growth rate of the algae that is being used for cultivation in the particular system.

In an operation for the alternate embodiment of the present invention, a transfer sequence is periodically performed in accordance with a set procedure. Specifically, the transfer sequence is initiated by first transferring fully grown algae from the last downstream pond to an oil formation pond, or directly to harvesting if desired. Once this is done, and the last downstream pond has been emptied, culture from the adjacent upstream pond is then transferred into the now-empty, last downstream pond. As the culture is transferred, additional medium can also be transferred into the last downstream pond for further algae growth in the last downstream pond. The now-empty, immediately upstream pond can then receive culture transferred from its respective adjacent upstream pond. This process of transfer from an upstream pond to an emptied adjacent downstream pond continues until the first upstream pond has been emptied and subsequently refilled with inoculum from the source of inoculum. After an entire transfer sequence has been completed, the cultures in all of the open ponds are individually circulated to promote algae growth. Once algae growth in the respective ponds has been completed, the entire transfer sequence can then be repeated. Preferably, transfer sequences for the alternate embodiment of the present invention are accomplished during the nighttime.

It is important to note that an open system as disclosed above can be effectively used for the purpose of preparing an algal culture for subsequent growth on a much larger scale. Specifically, for the commercial mass-production of a biomass, another open pond, bio-production system is needed. In particular, given an initial volume of algal culture which has been properly prepared, it is possible with a bio-production system to commercially grow an algal biomass having a volume that is many orders of magnitude greater than would be possible using a preparatory open system alone. The difference between the two systems is primarily due to the size of the respective systems, and the means that are used to mix and transport the algal culture during algal growth in the respective systems. With this in mind, the present invention recognizes that the two different systems are most efficient when used in combination with each other.

With regard to a smaller preparation system, during initial growth, an algal culture can be efficiently stirred in an open system by mechanical means (e.g. a paddle wheel drive). On the other hand, for a larger commercial scale, bio-production system, the enormity of such a system alone (e.g. up to around 50 acres of surface area for a single raceway pond) requires different considerations. For the present invention, this additional consideration tends toward a reliance on gravity for stirring and moving the algal culture as it progresses through the system.

For the present invention, once an initial volume of algal culture has grown to a sufficient size in the mechanically stirred ponds of a preparation system (e.g. ponds having approximately 200 to 400 m² of surface area), the initial volume of algal culture is then transferred to a sloped-gradient, gravity. driven, bio-production system. In this combination, an important aspect of the sloped-gradient, bio-production system is the ability to selectively move algal culture from one raceway pond to another pond, and to individually monitor growth parameters for the algal culture on a pond-by-pond basis. In particular, the bio-production system is designed to maintain constant algal growth rates, with constant algal concentration densities, at all times, in all of the different sloped-gradient, gravity-driven, raceway ponds of the bio-production system.

Structurally, an industrial scale, bio-production system for growing algae in accordance with the present invention includes a preparation system that is used to prepare an initial volume of algal culture. As intended for the present invention, each time an initial volume of algal culture has been grown in the preparation system, it is selectively transferred to a bio-production system. It is in the bio-production system where subsequent growth of the algal culture results in the mass production of an algal biomass.

In detail, the bio-production system includes a plurality of discrete raceway ponds, wherein each raceway pond is U-shaped to establish contiguous, parallel channels. Each raceway pond also has an upstream end and a downstream end with a predetermined sloped gradient along the length of the channels of the raceway pond. Further, each raceway pond has a unique predetermined surface area. For instance, as envisioned for the present invention, a predetermined surface area for the largest raceway pond in the sequence will be approximately fifty acres, or greater.

Depending on site conditions for the present invention, the predetermined sloped gradient for each raceway pond is established to promote an optimal growth rate for the algal culture and to also maintain a substantially constant fluid flow velocity through the raceway pond. From an operational perspective, the sloped gradient generates a linear fluid velocity for the algal culture through the raceway that is in a range between one and two feet per second.

Each raceway pond in the bio-production system also includes a partitioned sump with a lower sump for collecting algal culture from the downstream end of the raceway pond. A sump pump is also included for moving algal culture across the partition from the lower sump at the downstream end of the raceway pond to an upper sump at the upstream end of the raceway pond. After being transferred out of the upper sump, the algal culture that is collected in the upper sump is released into the raceway pond at its upstream end for further circulation of the algal culture through the particular raceway.

A sensor is provided for each raceway pond in the bio-production system. Specifically, the sensor is submerged in the upper sump of the raceway pond and it has various detectors for collecting algal growth parameter data. As envisioned for the present invention, this data includes measurements of temperature, pH, conductivity, turbidity, sump level, and algal cell concentration.

Control over each raceway pond in the bio-production system is provided by a pond control system. This pond control system is electronically connected with the sensors at each respective raceway pond for the purpose of real-time monitoring of the growth parameters of algal culture in the raceway pond. In addition to the real-time monitoring of the bio-production system using data provided from sensors in the raceway ponds, periodic off-line analyses can also be conducted to provide input for the pond control system. Further, weather information is monitored by the pond control system. Collectively, all this data can then be evaluated and assessed to determine an operational capability for the particular raceway pond. Further, this data can be transferred to a control module where it is evaluated and assessed with reference to data from other raceway ponds to determine an overall operational capability for the bio-production system.

An important feature of the present invention is a fluid transfer network that selectively connects each raceway pond of the bio-production system in fluid communication with every other pond in the system. Included within this fluid transfer network are a water source, a media source and a fertilizer source. Based on the specific assessment of the operational capability of a particular raceway pond or an overall assessment of the bio-production system, the various sources of additives for the system can be activated to simultaneously maintain substantially constant algal growth rates and substantially constant algal concentration densities throughout the bio production system.

As envisioned for the present invention, algal culture will be harvested typically from the raceway pond having the largest surface area Accordingly, a First-In-First-Out (FIFO) inventory management scheme is employed to insure an optimal turnover of algal culture in the system.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic view of the system of the present invention, illustrating the flow of algae from the closed reactor, through the expanding plug flow reactor, and to the standard plug flow reactor in accordance with the present invention;

FIG. 2 is an overhead view, riot to scale, of the expanding plug flow reactor shown in FIG. 1;

FIG. 3 is a longitudinal cross-sectional view of the expanding plug flow reactor of FIG. 2, showing the depth of the medium in the conduit;

FIG. 4 is a schematic view for an alternate embodiment of a system in accordance with the present invention;

FIG. 5 is a schematic representation of the present invention showing a mechanized algal culture preparation system in combination with a gravity-driven bio-production system in accordance with the present invention;

FIG. 6 is a top plan view of a representative raceway pond in the bio-production system;

FIG. 7 is a cross-section view of a channel for the raceway pond shown in FIG. 6 as would be seen along the line 7-7 in FIG. 6; and

FIG. 8 is a schematic representation of a bio-production system for the present invention showing a plurality of gravity-driven raceway ponds in combination with an interconnecting fluid transfer network and control capabilities.

### DESCRIPTION OF THE PREFERRED EMBODIMENTS

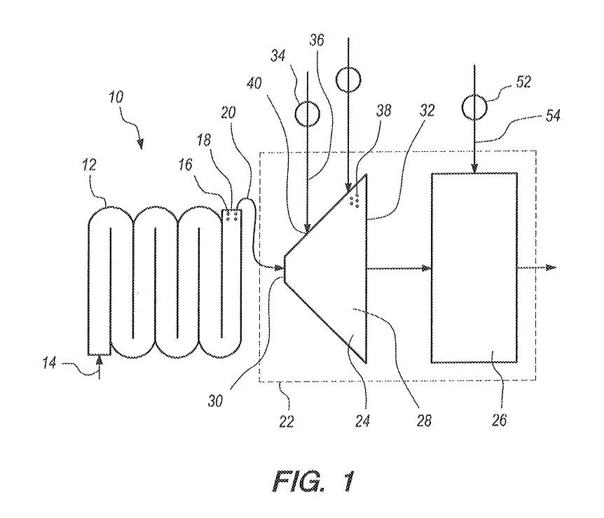

Referring initially to FIG. 1, a system for growing selected algae cells is shown, and is generally designated 10. As shown in FIG. 1, the system 10 includes a closed reactor 12, such as a continuous flow photobioreactor. As shown in FIG. 1, the closed reactor 12 is fed with an inoculum medium 14 and continuously grows an inoculum of algae 16. As the inoculum of algae 16 reaches the end 18 of the closed reactor 12, it is at full concentration. Then, the inoculum of algae 16 passes out of the closed reactor 12 in an effluence (arrow 20).

As shown in FIG. 1, the effluence 20 containing the inoculum of algae 16 passes from the closed reactor 12 to an open system 22, such as an open raceway. In FIG. 1, it can be seen that the open system 22 comprises an expanding plug flow reactor (EPFR) 24 and a standard plug flow reactor (SPFR) 26. Structurally, the EPFR 24 includes a conduit 28 with a first end 30 for receiving the effluence 20 and a second end 32. Further, the open system 22 includes a pump 34. As the effluence 20 enters the EPFR 24, the pump 34 adds a growth medium (arrow 36) to the EPFR 24 to dilute the concentration of algae 38 within the EPFR 24 to about 0.5 grams per liter of fluid. Further, the growth medium 36 includes the nutrients necessary to support the desired growth of the algae 38. As shown in FIG. 1, the open system 22 may include a plurality of pumps 34 for feeding the growth medium 36 at locations 40 along the length of the EPFR 24.

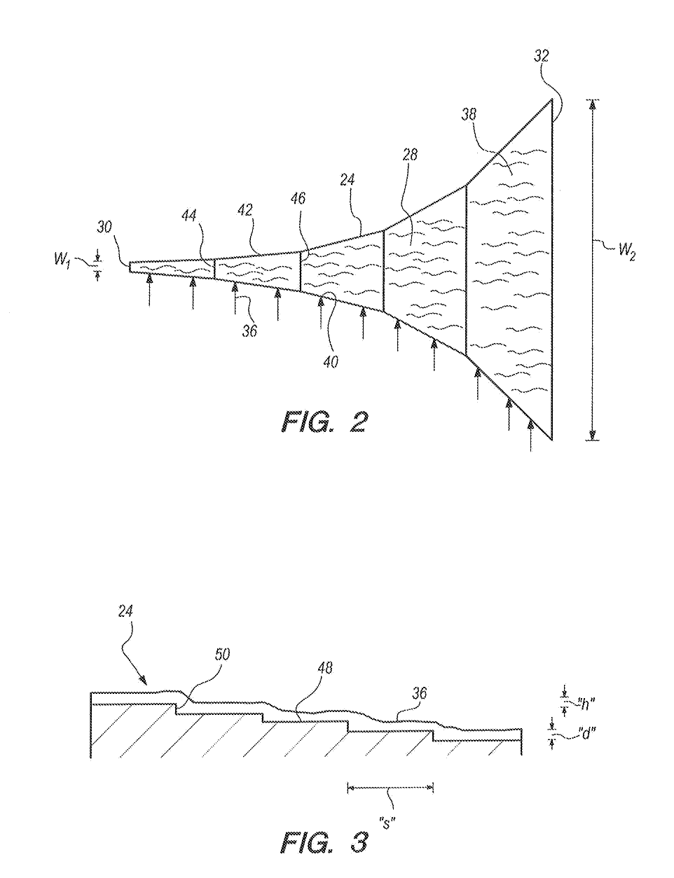

Referring now. to FIG. 2, the structure and operation of the EPFR 24 may be understood. As shown, the first end 30 of the EPFR 24 has a width W₁ and the second end 32 of the EPFR 24 has a width W₂ that is substantially greater than W₁. In FIG. 2, the EPFR 24 is not drawn to scale. In certain embodiments, W₁ will equal ten feet, while W₂ will equal 300 feet. Further, the EPFR 24 can be seen to include a plurality of sections 42. Further each section 42 expands in width from its proximal end 44 to its distal end 46. As shown, the width of each section 42 doubles from its proximal end 44 to its distal end 46. As a result, the EPFR 24 has a substantially logarithmic increase in width. While FIG. 2 illustrates an increase in width for each successive section, it is envisioned that sections 42 having a constant width could be interspersed among the widening sections 42.

Importantly, the fluid growth medium 36 and algae 38 flow through the EPFR 24 under the influence of gravity. For purposes of the present invention, this gravity flow is accomplished using a structured gradient. A preferred embodiment of a structured gradient for use with the EPFR 24 is shown in FIG. 3. There it will be seen that the floor 48 of the conduit 28 is formed with a plurality of steps 50. In detail, the steps 50 are defined by a height “h” of approximately 3 centimeters, with a distance “s” between the steps 50 being preferably on the order of approximately 100 meters. Typically, the EPFR 24 may be over 1000 meters long and the algae 38 may have a residence time of about thirty days in the EPFR 24.

An important aspect of the EPFR 24 for the present invention will be appreciated with reference to FIG. 3. This aspect is that the depth “d” of the fluid growth medium 36 in the conduit 28 needs to be rather shallow (i.e. less than about 15 cm, and preferably around 7.5 cm). To maintain this depth “d”, however, it is necessary to add the fluid growth medium 36 along the length of the EPFR 24 as the EPFR 24 widens, Importantly, the increase in width among EPFR sections 42 allows for logarithmic growth of the algae 38 while the concentration of the algae 38 is maintained at the high concentration of at least 0.5 grams per liter.

In cross-reference to FIGS. 1 and 2, as the growth medium 36 and algae 38 reach the second end 32 of the EPFR 24, they are transferred to the SPFR 26. At this stage, the algae 38 stops growing and, instead, begins to produce oils to store energy. In order to instigate oil production in the algae 38, a pump 52 may introduce a trigger medium 64 into the SPFR 26. Specifically, the trigger medium 54 may lack a desired nutrient, such as nitrogen or phosphorus, which causes the algae 38 to produce oil. Alternatively, the SPFR 26 may receive only the algae 38 from the EPFR 24, without any additional trigger medium 54. In either case, oil production in the algae 38 is triggered by the lack of nutrients to support growth.

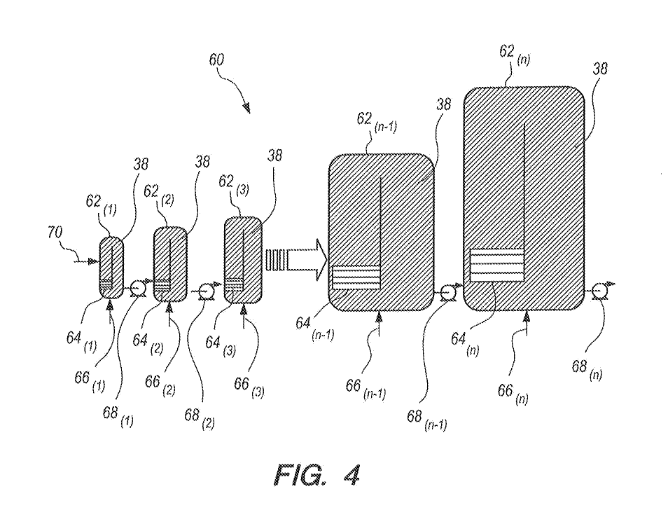

In FIG. 4, an alternate embodiment for the present invention is shown and is generally designated 60. As shown, the system 60 includes an “n” number of open ponds 62 with the smallest open pond 62₍₁₎ being designated as the “first upstream pond”, and the largest open pond 62₍ₙ₎ being designated as the “last downstream pond”. Intermediate open ponds 62 are arranged in order, according to size, with an exponentially increasing surface area in a downstream direction. In this case, the downstream direction extends from the first upstream pond 62₍₁₎ to the last downstream pond 62₍ₙ₎. For the system 60, the ratio between adjacent surface areas of respective open ponds 62 is established by a fixed multiplier. Importantly, this fixed multiplier is determined by the growth rate of the particular algae 38 that are to be cultivated in the system 60.

For the present invention, it is to be appreciated that all of the open ponds 62 in the system 60 are substantially similar to each other. The exception here is only in the size of their respective surface areas. Accordingly, each pond 62 will have a fluid circulating device 64 that is provided for moving (stirring) algae 38 around in the pond 62. Functionally, this is done to promote the growth of algae 38 while there is a culture of the algae 38 in the particular open pond 62. Examples for a suitable fluid circulating device 64 would be a standard circulation pump or a paddle wheel. Both of these types of devices are well known in the pertinent art.

It will also be seen in FIG. 4 that each open pond 62 has a medium addition conduit (represented by arrow 66) which is provided to add medium into the respective open pond 62, as needed. Further, the open ponds 62 are connected via respective transfer conduits for selective communication with each other. For example, the upstream open pond 62₍ₙ₋₁₎ is connected in fluid communication via a transfer conduit with its adjacent downstream open pond 62₍ₙ₎. Preferably, the transfer conduits are transfer pumps 68. As shown in FIG. 4, the transfer conduit between open pond 62₍ₙ₋₁₎ and open pond 62₍ₙ₎ is a transfer pump 68₍ₙ₋₁₎. As implied above, however, this particular structure is only exemplary. As an alternative to using transfer pumps 68, the open ponds 62 in system 60 can be terraced to provide for a gravity flow of liquid between the various pairs of upstream and downstream open ponds 62.

In addition to the specific structural components of the system 60 described above, inoculum algae 16 in an inoculum medium 14 can be fed into the first upstream open pond 62₍₁₎ via a first transfer conduit (represented by the arrow 70). At the downstream end of the system 60, after traversing the system 60, the now fully grown algae 38 can be removed from the last downstream open pond 62₍ₙ₎ via a last transfer conduit (e.g. transfer pump 68₍ₙ₎.

In the operation of the system 60, algae 38 are progressively grown as they are selectively passed from one open pond 62 to another. The actual time spent by the algae 38 in each open pond 62 in the series will be substantially the same, and will depend on the type of algae 38 that is being cultivated. As a practical matter, the time spent by algae 38 in a particular open pond 62 can be as much as several (e.g. 3) days. In the event, the transfer of algae 38 through the system 60 is done methodically. And preferably, the transfer will be accomplished at nighttime when the growth of algae 38 is delayed due to a lack of sun light.

A transfer sequence for moving algae 38 through the system 60 begins by first emptying the last downstream pond 62₍ₙ₎. To do this, the fully grown algae 38 therein are transferred through a transfer conduit (e.g. transfer pump 68₍ₙ₎) to an oil formation pond (i.e. SPFR 26). Next, the contents of the adjacent upstream open pond 62₍ₙ₋₁₎ are then emptied into the now-empty last downstream open pond 62₍ₙ₎. At this time, additional medium can be added to the last downstream open pond 62₍ₙ₎ via the medium addition conduit 66₍ₙ₎. Specifically, this is done to establish proper conditions for further growth of algae 38 in the open pond 62₍ₙ₎. In turn, the contents of open pond 62₍ₙ₋₂) (not shown) are emptied into open pond 62₍ₙ₋₁₎, and an appropriate amount of medium is added. This continues, in sequence, with the contents of each upstream open pond (e.g. pond 62₍₂₎) being transferred into the just-emptied adjacent downstream open pond (e.g. pond 62₍₃₎). The transfer sequence finally ends when the contents of the first upstream open pond 62₍₁₎ have been emptied into open pond 62₍₂₎ and the now-empty upstream open pond 62₍₁₎ has been refilled with inoculum of algae 16. The system 60 then continues to grow algae 38 in respective open ponds 62 until another transfer sequence is initiated.

The raceways depicted in FIG. 4 are not sloped and therefore require paddle wheels or similar motive force for mixing. Once a pond size reaches a wetted area of a maximum of approximately 1 acre, paddle wheels are no longer practical. A further increase in pond size necessitates transfer to a sloped pond system where all mixing is due to gravity-induced flow.

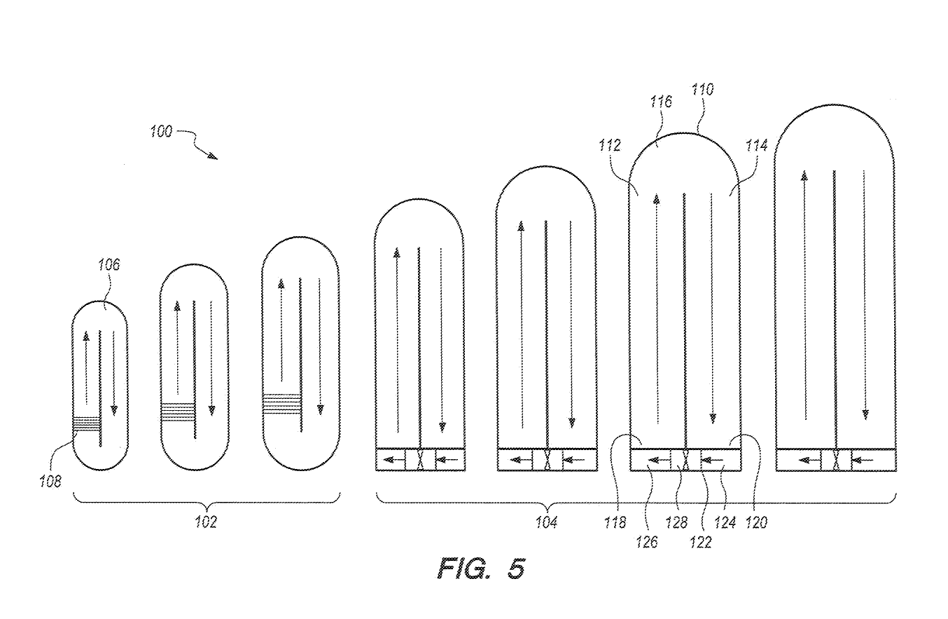

Referring now to FIG. 5, a system in accordance with the present invention is shown and is generally designated 100. As shown, the system 100 includes both a preparation system 102 and a bio-production system 104. There are several important differences, however, between the preparation system 102 and the bio-production system 104 when they are used as components of the system 100.

With reference to the preparation system 102, it is to be appreciated that the system 102 includes a plurality of similarly constructed open pond(s) 106. Essentially, the preparation system 102 is as disclosed above for the system 60 with reference to FIG. 4. In detail, each open pond 106 includes a mechanized stirring device, such as the paddle wheel 108 which is shown with the open pond designated 106 in FIG. 5. Structurally, all ponds 106 of the preparation system 102 are similar, with the exception of the size of their respective surface area. With this in mind, the ponds 106 are sequentially arranged in an order of increasingly larger exposed surface area. As disclosed above, the relationship between the ponds 106 in this sequence is established by a predetermined multiplier.

Operationally, the preparation system 102 is connected into fluid communication with the bio-production system 104 via a pumping means, such as the pump 68₍ₙ₎ disclosed above. FIG. 5 also shows that the bio-production system 104 includes a plurality of raceway ponds 110. Structurally, with the exception of the size of their respective surface area, all ponds 110 of the bio-production system 104 are similar. Like the ponds 106 of the preparation system 102 disclosed above, the raceway ponds 110 of the bio-production system 104 are sequentially arranged in an order of increasingly larger exposed surface area. Again, the relationship between the raceway ponds 110 in this sequence is established by the same predetermined multiplier that is used for the preparation system 102.

Still referring to FIG. 5, it is shown that each raceway pond 110 in the bio-production system 104 includes a fluid flow channel 112 and a fluid flow channel 114. In combination, the channels 112 and 114 are interconnected by a turn-around 116. As shown, the channels 112 and 114 are parallel to each other, they are contiguous, and they provide for a continuous fluid flow from the upstream end 118 of the raceway pond 110 to its downstream end 120. Further, FIG. 5 shows that raceway pond 110 includes a sump 122. In detail, the sump 122 has a lower sump 124 that is in fluid communication with the downstream end 120 of the raceway pond 110. It also has an upper sump 126 that is in fluid communication with the upstream end 118 of the raceway pond 110. Also included in the sump 122 is a pump 128 for transferring fluid (i.e. algal culture) from the lower sump 124 to the upper sump 126 for recirculation of the fluid (algal culture) through the raceway pond 110.

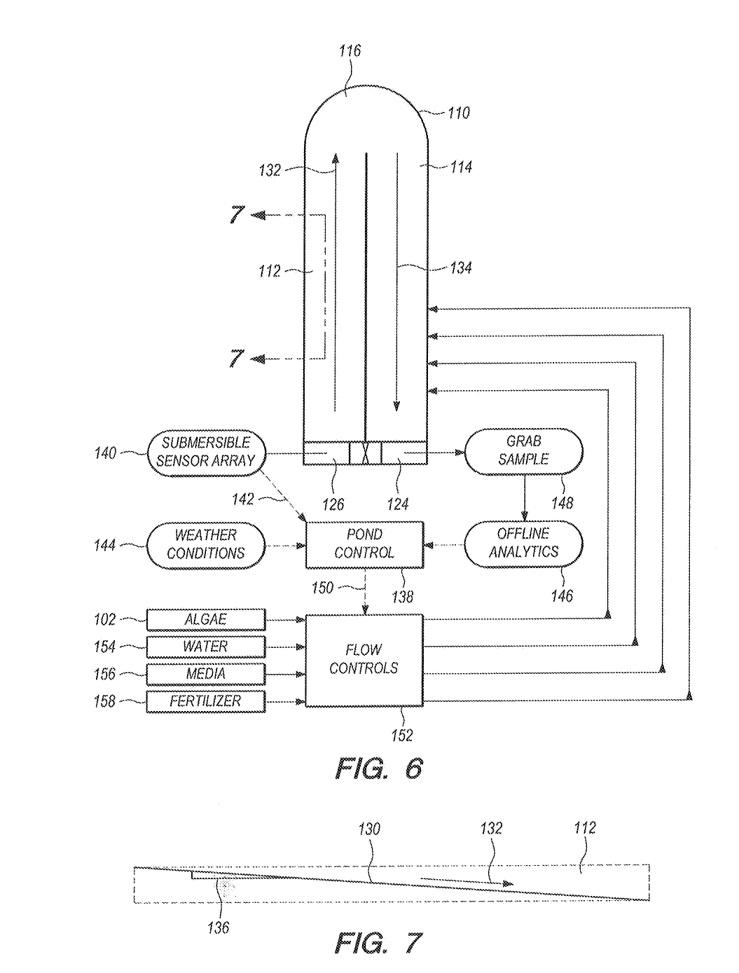

With cross reference to FIG. 6 and FIG. 7, it will be appreciated that fluid flow through the raceway pond 110 over the bottom 130 of the channels 112 and 114 will be in directions respectively indicated by the arrows 132 and 134. As specifically indicated in FIG. 7, the velocity of fluid flow through the channels 112 and 114 will be determined by a sloped gradient 136. For purposes of the present invention, the sloped gradient 136 is established to move the fluid (algal culture) at a linear fluid velocity through the raceway pond 110 that is in a range between one and two feet per second.

Referring now to FIG. 6, it will be seen that a pond control 138 is provided for the particular raceway pond 110. Recall, all raceway ponds 110 are essentially the same, structurally and functionally. With this in mind, the pond control 138 is electronically connected with a submersible sensor array 140 via a line 142. Further, the sensor array 140 is submerged in the upper sump 126 for collecting growth parameters of the algal culture in the pond 110. In particular, these growth parameters include: temperature, pH, conductivity, CO₂, turbidity, sump level, change in sump level and algal cell concentration. As they are being collected in real time, these growth parameters are transferred via the connecting line 142 to the pond control 138. FIG. 6 also shows that the pond control 138 receives input regarding weather conditions 144 as well as offline analytics 146, which can include a grab sample 148 that is taken from the lower sump 124. Together, all of this collected data is electronically transferred via line 150 from the pond control 138 to a set of flow controls 152.

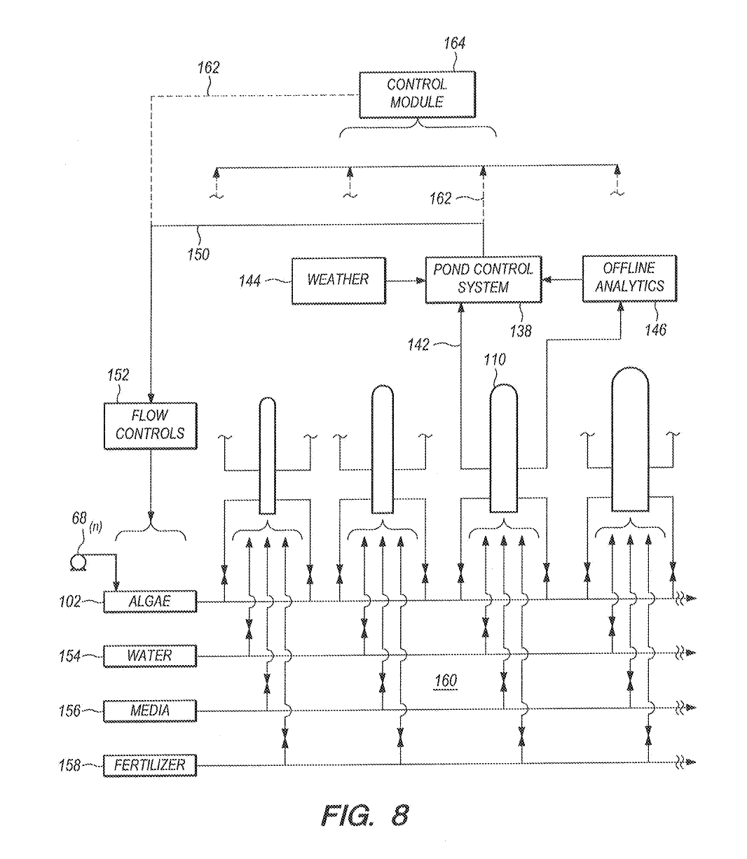

As shown in Both FIG. 6 and FIG. 8, the flow controls 152 are separately connected with an algal source (i.e. preparation system 102), a water source 154, a media source 156 and a fertilizer source 158. Importantly, the flow controls 152 are connected with a fluid transfer network 160. As presented in FIG. 8, the flow controls have effective operational control over the fluid transfer network 160. With this control, the preparation system 102 can be selectively connected in fluid communication with any raceway pond 110 in the bio-production system 104. Further, the water source 154, the media source 156, and the fertilizer source 158 can be selectively and individually connected in fluid communication with any raceway pond 110 in the bio-production system 104.

As intended for the present invention, via the fluid transfer network 160, the water source 154 can be used to supply water for maintaining a predetermined level of salinity, depth and cell density for algal culture in each individual raceway pond 110. Also, the media source 156 can provide a carbon source for instigating oil production in the algal culture in each individual raceway pond 110. And, the fertilizer source 158, can be activated to provide a supply of liquid fertilizer which will support the growth of algal culture in each of the individual raceway ponds 110. Moreover, via the fluid transfer network 160, individual raceway ponds 110 can be connected in fluid communication with each other.

As an added feature of the present invention, along with individual control over raceway ponds 110, the present invention envisions providing for an overall operational control of the entire system 100. In particular, as shown in FIG. 8, the dashed lines 162 show that a control module 164 can be incorporated to collect data directly from each of the individual raceway ponds 110. An analysis of this collected data can then be used by the control module 164 to activate the flow controls 152.

In addition to the normal routine testing and evaluation of algal culture in the individual raceway ponds 110, the fluid transfer network 160 also provides other operational capabilities. For instance, it may be necessary or desirable to empty a raceway pond 110, or a group of raceway ponds 110, for a particular purpose. If so, in accordance with the present invention, algal culture can be moved through the fluid transfer network 160 from a selected raceway pond(s) 110 having a relatively small surface area to another raceway pond 110 having a relatively larger surface area. The result in this example is that, by emptying the smaller raceway pond 110 into larger and temporarily deeper ponds, the system-wide surface area for the bio production system 104 is reduced to thereby minimize an impact from unexpected or undesirable events such as excessive rainfall. Transfers can also be made to allow for a re-inoculation of an empty raceway pond 110 or for redistributing algal culture in the plurality of raceway ponds 110 when a predetermined purpose has been completed.

While the particular Method and System for Growing Microalgae in Expanding Sloped Ponds as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A system for growing algae which comprises:
an open-pond, preparation system for growing an initial volume of an algal culture;
a bio-production system for receiving algal culture from the preparation system, the bio-production system including a plurality of discrete raceway ponds, wherein each raceway pond holds a respective volume of algal culture to simultaneously cultivate an algal biomass from the culture at a constant growth rate, to maintain a same constant concentration density for a same controlled residence time within each raceway pond, wherein each raceway pond is U-shaped to establish contiguous parallel channels, wherein each raceway pond has an upstream end and a downstream end with a predetermined sloped gradient therebetween, and wherein each raceway pond has a unique predetermined surface area;
a plurality of sumps, wherein each sump is connected to a respective raceway pond and is partitioned to have a lower sump in fluid communication with the downstream end of the raceway pond, and an upper sump in fluid communication with the upstream end of the raceway pond, and wherein the sump includes a pump for transferring algal culture from the lower sump to the upper sump for recirculation of the algal culture through the raceway;
a plurality of sensors, wherein each sensor is submerged in algal culture in the upper sump of a respective raceway pond to collect algal growth parameter data from algal culture in the raceway pond; and
a pond control system electronically connected with the plurality of submerged sensors in the respective raceway pond to monitor and evaluate the algal growth parameter data therein, in order to implement corrective actions necessary to maintain constant growth rates and constant algal culture concentration densities in the raceway pond of the bio-production system.

2. The system recited in claim 1 further comprising a fluid transfer network interconnecting each raceway pond in fluid communication with at least one other raceway pond.

3. The system recited in claim 2 wherein the fluid transfer network further comprises:
a water source containing necessary nutrients for maintaining a predetermined level of salinity, depth and cell density for algal culture in each individual raceway pond;
a media source for instigating oil production in the algal culture in each individual raceway pond; and
a fertilizer source for supporting a growth of algal culture in each individual raceway pond.

4. The system recited in claim 3 wherein the growth parameters include temperature, pH, conductivity, CO₂, turbidity, sump level, change in sump level, and algal cell concentration.

5. The system recited in claim 4 wherein a target for the concentration density of algal cells is a range between 0.5 and 1 gram per liter.

6. The system recited in claim 1, wherein the plurality of raceway ponds is sequentially organized according to an increase in the respective predetermined surface area of each pond in the plurality, and wherein the sequential increase is established in accordance with a multiplier, wherein the multiplier accounts for algae growth factors identified for the system, and wherein the multiplier relates the predetermined surface area of each pond in the sequence to a predetermined surface area of an immediately adjacent pond in the sequence.

7. The system recited in claim 6 wherein the predetermined surface area of the largest raceway pond in the sequence is fifty acres.

8. The system recited in claim 1 wherein the sloped gradient of each raceway pond generates a linear fluid velocity for the algal culture in a range between one and two feet per second and wherein algal culture is harvested from the raceway pond having the largest surface area.

9. The system recited in claim 1 further comprising a control module connected to each pond control system to determine an overall operational capability of the bio-production system.

10. The system recited in claim 1 wherein the preparation system comprises:
a plurality of open ponds, wherein the open ponds are arranged in sequential order according to size, with an exponentially increasing surface area in one direction;
a means individually provided for each pond in the sequence for stirring the algal culture in the respective open pond; and
a pump for transferring algal culture from the preparation system to the plurality of open raceway ponds.

11. The system recited in claim 10 wherein the algal culture is transferred from a last open pond in the sequential order, and the last open pond has a surface area in a range between 400 and 4,000 m².

12. A method for using a bio-production system for growing algae which comprises the steps of:
providing a preparation system comprising an open pond reactor for growing an initial volume of an algal culture, a plurality of discrete raceway ponds in the bio-production system for sequentially receiving a respective volume of algal culture from the preparation system to simultaneously cultivate an algal biomass from the culture at a constant growth rate, to maintain a same constant concentration density for a same controlled residence time within each raceway pond, wherein each raceway pond is U-shaped to establish contiguous parallel channels, wherein each raceway pond has an upstream end and a downstream end with a predetermined sloped gradient therebetween, and wherein each raceway pond has a unique predetermined surface area, a sensor submerged in the algal culture in each raceway pond to collect algal growth parameter data, a pond control system to monitor and evaluate the algal growth parameter data, and a fluid transfer network interconnecting each raceway pond in fluid communication with at least one other raceway pond;
connecting a water source, a media source, and a fertilizer source into respective fluid communication with the fluid transfer network of the bio-production system;
implementing corrective actions to maintain constant growth rates and constant algal concentration densities in each raceway pond; and
configuring the fluid transfer network to achieve a predetermined fluid flow pattern within the bio-production system required for the implementing step.

13. The method recited in claim 12 wherein the growth parameters include temperature, pH, conductivity, CO₂, turbidity, sump level, change in sump level, and algal cell concentration.

14. The method recited in claim 12 wherein the implementing step is accomplished by moving water from the water source containing necessary nutrients to maintain a predetermined level of salinity, depth and cell density for algal culture in each individual raceway pond.

15. The method recited in claim 12 wherein the implementing step is accomplished by moving media from the media source to instigate oil production in the algal culture of each individual raceway pond.

16. The method recited in claim 12 wherein the implementing step is accomplished by moving fertilizer from the fertilizer source to support a growth of algal culture in each individual raceway pond.

17. The method recited in claim 12 further comprising the step of moving algal culture through the fluid transfer network from a pond having a relatively small surface area to a pond having a relatively larger surface area to empty the small pond for a predetermined purpose.

18. The method recited in claim 17 wherein the moving step is accomplished to reduce the system-wide surface area to minimize an impact from adverse weather conditions.

19. The method recited in claim 17 wherein the moving step is accomplished to allow for a re-inoculation of the empty pond.

20. The method recited in claim 17 further comprising the step of redistributing algal culture in the plurality of raceway ponds when the predetermined purpose is completed.
